(19) United States
(12) Patent Application Publication
(10) Pub. No.: US 2025/0309381 A1
(43) Pub. Date: Oct. 2, 2025

(54) GAS DETECTION UNIT, BATTERY PACK, GAS DETECTION SYSTEM, BATTERY SYSTEM AND GAS DETECTION METHOD

(71) Applicant: TOYOTA JIDOSHA KABUSHIKI KAISHA, Toyota-shi (JP)

(72) Inventors: Yasumasa OGUMA, Nagoya-shi (JP); Shigeyuki MORI, Nagoya-shi (JP)

(73) Assignee: TOYOTA JIDOSHA KABUSHIKI KAISHA, Toyota-shi (JP)

(21) Appl. No.: 19/046,685

(22) Filed: Feb. 6, 2025

(30) Foreign Application Priority Data
Apr. 2, 2024 (JP) 2024-059774

(51) Int. Cl. H01M 10/42; G01N 33/00; H01M 50/202

(52) U.S. Cl. CPC H01M 10/4285; G01N 33/0044; H01M 50/202; H01M 2220/20; H01M 2300/0068

(57) ABSTRACT

The disclosure provides a gas detection unit that can detect hydrogen sulfide-containing gas with high precision, a battery pack comprising the detection unit, a gas detection system, a battery system comprising the gas detection system, and a gas detection method. The gas detection unit 10 of the disclosure comprises a hydrogen sulfide adsorbent 11, a heating section 12 that heats the hydrogen sulfide adsorbent, and a gas detection section 13 that detects the hydrogen sulfide and/or sulfur-containing gas released from the hydrogen sulfide adsorbent. The battery pack 1 of the disclosure comprises a sulfide-based battery 20, a gas detection unit 10 of the disclosure, and an outer container 30 that houses the sulfide-based battery 20 and gas detection unit 10.

### FIELD

The present disclosure relates to a gas detection unit, a battery pack, a gas detection system, a battery system and a gas detection method.

### BACKGROUND

In a sulfide-based battery comprising a sulfide-based compound, the sulfide-based compound may react with water to form hydrogen sulfide. Techniques have been developed for detecting such generated hydrogen sulfide.

For example, PTL 1 discloses a battery system provided with a battery pack, a gas sensor that detects the concentration of hydrogen sulfide-containing gas in the battery pack, a temperature sensor that detects the temperature inside the battery pack, and an abnormality diagnosing apparatus that diagnoses that an abnormally high concentration of hydrogen sulfide has been generated in the battery pack when the concentration detected by the gas sensor is higher than a threshold concentration, wherein the abnormality diagnosing apparatus increases the threshold concentration as the temperature in the battery pack increases.

In other words, PTL 1 discloses that increasing the threshold of hydrogen sulfide concentration for diagnosing that an abnormally high concentration of hydrogen sulfide has been generated in response to increasing temperature inside the battery pack.

### CITATION LIST

### Patent Literature

[PTL 1] Japanese Unexamined Patent Publication No. 2022-012308

### SUMMARY

### Technical Problem

Increasing the threshold of the hydrogen sulfide concentration may result in failing to detect hydrogen sulfide even when hydrogen sulfide is in fact generated. It is preferable to be able to detect hydrogen sulfide-containing gas with high precision.

It is an object of the disclosure to provide a gas detection unit that can detect hydrogen sulfide-containing gas with high precision, and a battery pack comprising the detection unit. It is another object of the disclosure to provide a gas detection system that can detect hydrogen sulfide-containing gas with high precision, and a battery system comprising the gas detection system. It is yet another object of the disclosure to provide a gas detection method that allows detection of hydrogen sulfide-containing gas with high precision.

### Solution to Problem

The present inventors have found that the aforementioned object can be achieved by the following means.

### <Aspect 1>

A gas detection unit comprising:
a hydrogen sulfide adsorbent that adsorbs hydrogen sulfide and releases the hydrogen sulfide and/or sulfur-containing gas derived from the hydrogen sulfide at or above a predetermined temperature,
a heating section that heats the hydrogen sulfide adsorbent, and
a gas detection section that detects the hydrogen sulfide and/or sulfur-containing gas released from the hydrogen sulfide adsorbent.

### <Aspect 2>

The gas detection unit according to aspect 1, wherein the gas detection section is disposed above the hydrogen sulfide adsorbent.

### <Aspect 3>

The gas detection unit according to aspect 1 or 2, wherein the heating section heats the hydrogen sulfide adsorbent at a temperature of 100° C. or higher and 400° C. or lower.

### <Aspect 4>

A battery pack comprising a sulfide-based battery, a gas detection unit according to any one of aspects 1 to 3, and an outer container that houses the sulfide-based battery and gas detection unit,
wherein the hydrogen sulfide is generated from the sulfide-based battery.

### <Aspect 5>

The battery pack according to aspect 4, wherein the hydrogen sulfide adsorbent is disposed below the sulfide-based battery.

### <Aspect 6>

A gas detection system comprising:
a gas detection unit according to any one of aspects 1 to 3, and
a control unit that actuates the heating section at a predetermined interval.

### <Aspect 7>

The gas detection system according to aspect 6, wherein:
the gas detection system further comprises a notification unit,
the control unit actuates the notification unit when the concentration of the hydrogen sulfide and/or sulfur-containing gas detected by the gas detection section is higher than a threshold.

### <Aspect 8>

A battery system comprising a sulfide-based battery and a gas detection system according to aspect 6 or 7,
wherein the hydrogen sulfide is generated from the sulfide-based battery.

### <Aspect 9>

A gas detection method comprising the following steps:
adsorbing hydrogen sulfide with a hydrogen sulfide adsorbent,
heating the hydrogen sulfide adsorbent with a heating section to cause release of the hydrogen sulfide and/or sulfur-containing gas derived from the hydrogen sulfide, from the hydrogen sulfide adsorbent, and
detecting the hydrogen sulfide and/or sulfur-containing gas released from the hydrogen sulfide adsorbent, with a gas detection section.

### <Aspect 10>

The gas detection method according to aspect 9, wherein a hydrogen sulfide adsorption device is disposed in an outer container of a battery pack together with a sulfide-based battery, causing hydrogen sulfide generated from the sulfide-based battery to be adsorbed by the hydrogen sulfide adsorption device.

### Advantageous Effects of Invention

According to the disclosure it is possible to provide a gas detection unit that can detect hydrogen sulfide-containing gas with high precision, and a battery pack comprising the detection unit. According to the disclosure it is also possible to provide a gas detection system that can detect hydrogen sulfide-containing gas with high precision, and a battery system comprising the gas detection system. According to the gas detection method of the disclosure it is possible to detect hydrogen sulfide-containing gas with high precision.

### BRIEF DESCRIPTION OF DRAWINGS

FIG. 1 is a schematic diagram showing an example of the gas detection unit of the disclosure.

FIG. 2 is a schematic diagram showing an example of a battery pack of the disclosure.

FIG. 3 is a schematic diagram showing the overall construction of a gas detection system of the disclosure.

FIG. 4 is a flow chart showing the processing carried out by a gas detection system and battery system of the disclosure.

FIG. 5 is a schematic diagram showing the overall construction of a battery system of the disclosure.

### DESCRIPTION OF EMBODIMENTS

An embodiment of the disclosure will now be described in detail with reference to the accompanying drawings. The disclosure is not limited to the embodiment described below, however, and various modifications may be implemented which do not depart from the gist thereof. The dimensional relationships in the drawings do not reflect actual dimensional relationships.

### <<Gas Detection Unit>>

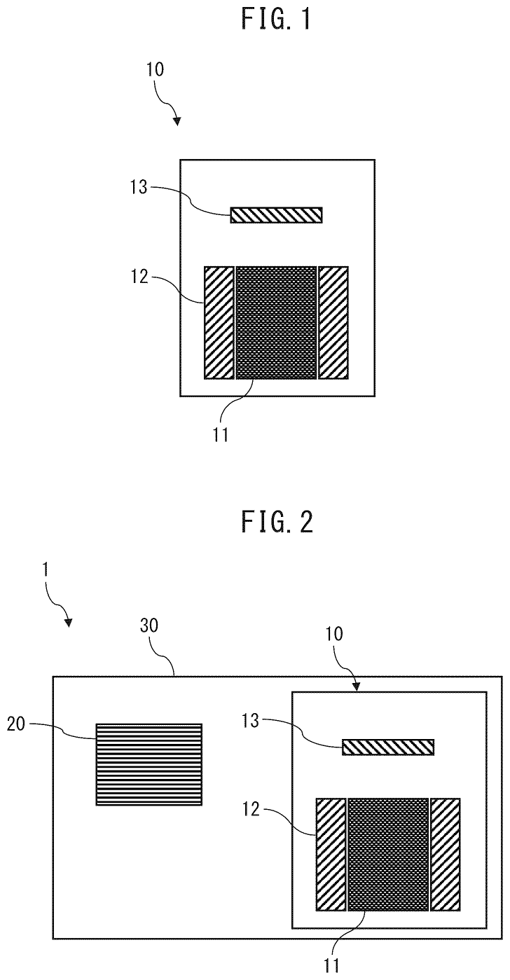

As shown in FIG. 1, the gas detection unit 10 of the disclosure comprises a hydrogen sulfide adsorbent 11 that adsorbs hydrogen sulfide and releases the hydrogen sulfide and/or sulfur-containing gas derived from the hydrogen sulfide at or above a predetermined temperature, a heating section 12 that heats the hydrogen sulfide adsorbent, and a gas detection section 13 that detects the hydrogen sulfide and/or sulfur-containing gas released from the hydrogen sulfide adsorbent.

When attempting to detect hydrogen sulfide, it may be impossible to properly detect hydrogen sulfide. The present inventors considered that one reason for this is low gas selectivity for detecting hydrogen sulfide by the gas detection section, which is often a gas sensor, such that when gases other than hydrogen sulfide are present, generation of hydrogen sulfide may be falsely detected even if no hydrogen sulfide is actually being generated. In order to prevent such false detection, it is necessary to set a higher threshold for the concentration at which hydrogen sulfide is detected by the gas detection section, but when this is done, it may not be possible to detect hydrogen sulfide even when hydrogen sulfide is actually being generated.

The present inventors have found that by heating a hydrogen sulfide adsorbent to release hydrogen sulfide adsorbed by the hydrogen sulfide adsorbent, and detecting the released hydrogen sulfide with a gas detection section, it is possible to cause highly concentrated hydrogen sulfide to contact with the gas detection section and thus allow detection of hydrogen sulfide-containing gas with high precision. Since heating of hydrogen sulfide may generate sulfur-containing gases other than hydrogen sulfide, such as sulfur oxides, the gas detection section may be one that can detect such sulfur-containing gases as well.

### <Hydrogen Sulfide Adsorbent>

As shown in FIG. 1, the gas detection unit 10 of the disclosure may comprise a hydrogen sulfide adsorbent 11. The hydrogen sulfide adsorbent 11 adsorbs hydrogen sulfide and releases hydrogen sulfide and/or sulfur-containing gas derived from hydrogen sulfide, at or above a predetermined temperature.

Examples of hydrogen sulfide adsorbents include activated carbon, alumina and zeolite, as matrix materials. These hydrogen sulfide adsorbents may also be supported on a carrier. Examples of carriers include Ag, KMnO₄, NaMnO₄, K₂CO₃, NaHCO₃ and CuO. Such hydrogen sulfide adsorbents in powdered form may also be used after solidification into pellets.

The predetermined temperature will differ depending on the type of hydrogen sulfide adsorbent. When the hydrogen sulfide adsorbent is activated carbon, for example, the predetermined temperature may be 100° C.

The sulfur-containing gas derived from hydrogen sulfide is not particularly restricted so long as it is a gas released from the hydrogen sulfide adsorbent and contains sulfur element, and examples include sulfur oxides such as sulfur dioxide.

The hydrogen sulfide adsorbent may be housed in a container having an opening.

### <Heating Section>

As shown in FIG. 1, the gas detection unit 10 of the disclosure comprises a heating section 12. The heating section 12 heats the hydrogen sulfide adsorbent 11. The hydrogen sulfide adsorbent 11 that has been heated at or above the predetermined temperature by the heating section 12 releases hydrogen sulfide and/or sulfur-containing gas derived from hydrogen sulfide.

The heating section is not particularly restricted so long as it can heat the hydrogen sulfide adsorbent, and it may be a heater, for example.

The heating section 12 may heat the hydrogen sulfide adsorbent 11 at a temperature of 100° C. or higher and 400° C. or lower.

Particularly if the hydrogen sulfide adsorbent is activated carbon, it will be possible to effectively release hydrogen sulfide and/or sulfur-containing gas at a temperature of 100° C. or higher and 400° C. or lower. Activated carbon also as a low level of decomposition in the temperature range of 100° C. or higher and 400° C. or lower, and can therefore be repeatedly used as a hydrogen sulfide adsorbent, thus reducing material costs and costs required for replacement.

The location of the heating section is not particularly restricted so long as it is a location that allows heating of the hydrogen sulfide adsorbent. In FIG. 1, the heating section is shown disposed at both ends of the hydrogen sulfide adsorbent for illustration, but the location of the heating section is not limited to the illustration.

### <Gas Detection Section>

As shown in FIG. 1, the gas detection unit 10 of the disclosure comprises a gas detection section 13. The gas detection section 13 detects hydrogen sulfide and/or sulfur-containing gas that has been released from the hydrogen sulfide adsorbent 11.

The gas detection section is not particularly restricted so long as it can detect hydrogen sulfide and/or sulfur-containing gas. The gas detection section may be a gas sensor, for example.

The gas detection section 13 may be disposed in a predetermined flow passage for the released hydrogen sulfide and/or sulfur-containing gas. For example, the gas detection section 13 may be disposed above the hydrogen sulfide adsorbent 11. Since heated gas tends to move upwards, this construction facilitates contact between the gas detection section 13 and the hydrogen sulfide and/or sulfur-containing gas that has been released from the hydrogen sulfide adsorbent 11 by the heating, thus aiding in detection of hydrogen sulfide and/or sulfur-containing gas by the gas detection section 13. This can reduce production costs since it eliminates the need for a ventilation path to direct the released hydrogen sulfide and/or sulfur-containing gas to the gas detection section 13. When the hydrogen sulfide adsorbent is housed in a container with an opening, for example, the gas detection section may be disposed above, close to the opening.

### <<Battery Pack>>

As shown in FIG. 2, the battery pack 1 of the disclosure comprises a sulfide-based battery 20, a gas detection unit 10 of the disclosure, and an outer container 30 that houses the sulfide-based battery 20 and gas detection unit 10. In the battery pack 1 of the disclosure, the hydrogen sulfide adsorbed by the hydrogen sulfide adsorbent 11 is generated from the sulfide-based battery 20. In other words, the battery pack 1 of the disclosure comprises a sulfide-based battery 20, a gas detection unit 10, and an outer container 30 housing the sulfide-based battery 20 and gas detection unit 10, while the gas detection unit 10 comprises a hydrogen sulfide adsorbent 11 that adsorbs hydrogen sulfide generated from the sulfide-based battery 20 and releases the hydrogen sulfide and/or sulfur-containing gas derived from hydrogen sulfide at or above the predetermined temperature, a heating section 12 that heats the hydrogen sulfide adsorbent 11, and a gas detection section 13 that detects the hydrogen sulfide and/or sulfur-containing gas that has been released from the hydrogen sulfide adsorbent 11. Since the battery pack 1 of the disclosure comprises the gas detection unit 10 of the disclosure, it can detect with high precision hydrogen sulfide-containing gas that can sometimes be generated from the sulfide-based battery 20.

### <Sulfide-Based Battery>

As shown in FIG. 2, the battery pack 1 of the disclosure comprises a sulfide-based battery 20. The term “sulfide-based battery” as used herein means a battery that includes a sulfide-based compound. The sulfide-based compound is not particularly restricted, and may be a sulfide-based solid electrolyte, for example. The sulfide-based battery 20 will sometimes generate hydrogen sulfide due to reaction between the sulfide-based compound and moisture. Examples of moisture that reacts with sulfide-based compounds include moisture in external air that has infiltrated into the outer container 30, and water that has infiltrated into the outer container 30.

The sulfide-based battery may be a liquid battery or a solid-state battery. The sulfide-based battery may also include a sulfide-based compound such as a sulfide solid electrolyte in one or more from among a positive electrode active material layer, electrolyte layer and negative electrode active material layer. The sulfide-based battery is most preferably a sulfide solid-state battery comprising a sulfide solid electrolyte in the solid electrolyte layer.

The term “solid-state battery” as used herein refers to a battery using at least a solid electrolyte as the electrolyte, and the solid-state battery may employ a combination of a solid electrolyte and a liquid electrolyte as the electrolyte. The solid-state battery of the disclosure may also be an all-solid-state battery, i.e. a battery employing only a solid electrolyte as the electrolyte.

The sulfide-based battery may be a primary battery, or a secondary battery such as a lithium ion battery or sodium ion battery.

When the sulfide-based battery of the disclosure is a lithium ion battery, examples of sulfide solid electrolytes may include, but are not limited to, sulfide amorphous solid electrolytes, sulfide crystalline solid electrolytes and argyrodite solid electrolytes. Specific examples of sulfide solid electrolytes include, but are not limited to, Li₂S—P₂S₅ (Li₇P₃S₁₁, Li₃PS₄, Li₈P₂S₉), Li₂S—SiS₂, LiI—Li₂S—SiS₂, LiI—Li₂S—P₂S₅, LiI—LiBr—Li₂S—P₂S₅, Li₂S—P₂S₅—GeS₂ (Li₁₃GeP₃S₁₆, Li₁₀GeP₂S₁₂), LiI—Li₂S—P₂O₅, LiI—Li₃PO₄—P₂S₅ and Li₇₋ₓPS₆₋ₓClₓ, as well as combinations thereof.

When the sulfide-based battery of the disclosure is a sodium ion battery, examples of sulfide solid electrolytes may include, but are not limited to, Na₂S—P₂S₅ such as Na₃PS₄, or Na₃SbS₄ and Na_{2.88}Sb_{0.88}W_{0.12}S₄, or their combinations.

A sulfide solid electrolyte may be glass or crystallized glass (glass ceramic).

According to the disclosure, the sulfide-based battery may be a “laminated battery” (also known as “pouch battery”), as a battery having an electrode stack with a negative electrode collector layer, a negative electrode active material layer, an electrolyte layer, a positive electrode active material layer and a positive electrode collector layer stacked in that order, housed inside a laminate film as the battery-housing case. When the sulfide-based battery is a laminated battery, opening of the sealed sections of the laminate film tends to occur, allowing contact between the sulfur-based compound and moisture, and tending to result in hydrogen sulfide generation from the sulfide-based battery. The sulfide-based battery of the disclosure is therefore particularly useful as a laminated battery.

The case housing the sulfide-based battery does not need to be a laminate film. That is, the sulfide-based battery may be a type of battery other than a laminated battery, such as a rectilinear, cylindrical or coin battery.

The materials for the negative electrode collector layer, negative electrode active material layer, electrolyte layer, positive electrode active material layer and positive electrode collector layer may be used as appropriate and common for materials used for such layers in a sulfide-based battery.

The shape and size of the electrode stack is not particularly restricted.

The shape, size and material of the case housing the battery are also not particularly restricted.

The sulfide-based battery housed in an outer container as described below is electrically connected in a serial or parallel manner, or in a combination of serial and parallel. The number of sulfide-based batteries housed in an outer container may be one. The sulfide-based battery may also be a battery module composed of two or more unit cells. In this case, the unit cells in each battery may be electrically connected in a serial manner or parallel manner, or in a combination of serial and parallel. The placement of the battery or batteries in the outer container is not particularly restricted and may be any desired placement. A plurality of batteries may be stacked together or disposed separately from each other.

The method for producing the sulfide-based battery is not particularly restricted, and it may be produced by a conventional publicly known method.

### <Gas Detection Unit>

As shown in FIG. 2, the battery pack 1 of the disclosure comprises a gas detection unit 10 of the disclosure. The gas detection unit 10 of the disclosure will be understood by referring to the description herein regarding the gas detection unit of the disclosure.

The hydrogen sulfide adsorbent 11 in the gas detection unit 10 may be disposed below the sulfide-based battery 20. Since the specific gravity of hydrogen sulfide is greater than the specific gravity of air, such a construction helps the hydrogen sulfide adsorbent 11 to adsorb hydrogen sulfide generated from the sulfide-based battery 20. In particular, the hydrogen sulfide adsorbent 11 may be disposed at the bottom of the battery pack 1.

### <Outer Container>

As shown in FIG. 2, the battery pack 1 of the disclosure comprises an outer container 30. The outer container 30 houses the sulfide-based battery 20 and the gas detection unit 10.

The shape and size of the outer container are not particularly restricted so long as the container is able to house the sulfide-based battery and hydrogen sulfide adsorbent.

The material forming the outer container is also not particularly restricted and may be one commonly used for outer containers of battery packs. Such materials include metals, and specifically aluminum and stainless steel.

### <<Gas Detection System>>

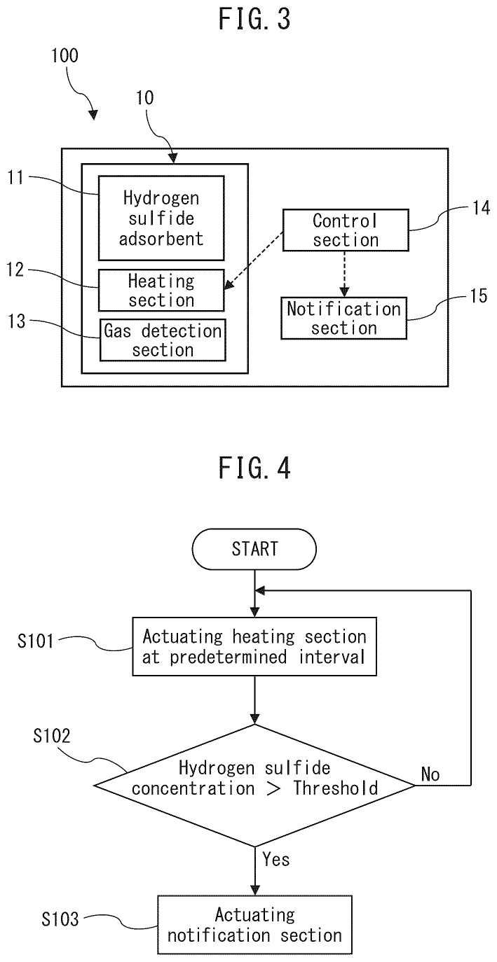

As shown in FIG. 3, the gas detection system 100 of the disclosure comprises a gas detection unit 10 of the disclosure, and a control unit 14 that actuates the heating section at a predetermined interval. Specifically, the gas detection system 100 of the disclosure comprises a hydrogen sulfide adsorbent 11 that adsorbs hydrogen sulfide and releases hydrogen sulfide and/or sulfur-containing gas derived from hydrogen sulfide at or above a predetermined temperature, a heating section 12 that heats the hydrogen sulfide adsorbent, a gas detection section 13 that detects hydrogen sulfide and/or sulfur-containing gas released from the hydrogen sulfide adsorbent, and a control unit 14 that actuates the heating section at the predetermined interval. With such a construction it is possible to detect hydrogen sulfide-containing gas with high precision, and to reduce labor and cost for manual operation of the heating section. It is also possible to prevent excess adsorption of hydrogen sulfide onto the hydrogen sulfide adsorbent.

### <Gas Detection Unit>

The gas detection unit 10 of the gas detection system 100 of the disclosure will be understood by referring to the description herein regarding the gas detection unit of the disclosure.

### <Control Unit>

As shown in FIG. 3, the gas detection system 100 of the disclosure comprises a control unit 14. The control unit 14 actuates the heating section 12 at the predetermined interval. The predetermined interval can be appropriately designed in consideration of the hydrogen sulfide adsorption capacity and amount of use of the hydrogen sulfide adsorbent 11.

The control unit 14 may be a processor such as a CPU (Central Processing Unit). The gas detection system of the disclosure may therefore include a CPU, and may further include a memory, input/output ports for input and output of signals, and a sensor. The CPU may carry out various control operations based on signals received from a sensor, and on a program stored in memory. The memory may be a volatile semiconductor memory (such as RAM), a non-volatile semiconductor memory (such as ROM), a hard disk drive (HDD), a solid state drive (SSD) or an optical recording medium.

By prestoring in memory a program relating to the predetermined interval for actuation of the heating section, for example, the CPU can control actuation of the heating section at the predetermined interval based on the program. More specifically, the gas detection system of the disclosure may also include a timer, allowing the CPU to control actuation of the heating section based on a signal inputted from the timer, and a program associated with the predetermined interval, which is prestored in the memory.

As a different embodiment, the control unit may carry out control for actuation of the heating section when a given parameter, such as pressure inside the outer container, has reached or exceeded a threshold value. The method for measuring the given parameter in the outer container is not particularly restricted, and it may be a method of measurement by a detecting unit such as a sensor. Specifically, when the control unit is to cause actuation of the heating section based on the pressure inside the outer container, the battery pack of the disclosure may further comprise a pressure detecting unit such as a pressure sensor. The pressure inside the outer container in this case may be constantly monitored using the pressure detecting unit. The threshold for the pressure inside the outer container is not particularly restricted so long as it is a value allowing judgment of increase in the pressure as hydrogen sulfide is generated. When the detected pressure inside the outer container is at or above the threshold value, the pressure detecting unit generates a signal, the signal being inputted to the CPU acting as the control unit, and allowing the CPU to control actuation of the heating section based on the signal.

### <Notification Unit>

The gas detection system 100 of the disclosure further comprises a notification unit 15. The control unit 14 may actuate the notification unit 15 when the concentration of the hydrogen sulfide and/or sulfur-containing gas as detected by the gas detection section 13 is higher than the threshold. That is, the notification unit 15 may be activated by the control unit 14, to notify when the concentration of the hydrogen sulfide and/or sulfur-containing gas as detected by the gas detection section 13 is higher than the threshold. For example, when the detected hydrogen sulfide concentration is higher than the threshold, the gas detection section 13 may generate a signal, the signal being inputted into the CPU acting as the control unit 14, and allowing it to actuate the notification unit 15 based on the signal. A program that halts control by the control unit 14 for actuation of the heating section 12 when the signal has been inputted into the CPU may also be prestored in the memory, with the control unit 14 actuating the notification unit 14 based on the program and subsequently halting control of actuation of the heating section 12.

The notification unit 15 is not particularly restricted and may be an indicator light or speaker, for example.

Processing carried out by the gas detection system 100 of the disclosure will now be explained with reference to the flow chart shown in FIG. 4.

As shown in FIG. 4, in the gas detection system 100 of the disclosure, first the control unit 14 actuates the heating section 12 at the predetermined interval (step S101).

Based on the signal inputted from the gas detection section 13, the control unit 14 then compares the concentration of hydrogen sulfide and/or sulfur-containing gas detected by the gas detection section 13, with the preset threshold (step S102). When the concentration of the hydrogen sulfide and/or sulfur-containing gas is at or below the threshold, the control unit 14 repeats step S101 and step S102.

When the concentration of the hydrogen sulfide and/or sulfur-containing gas is above the threshold, on the other hand, the control unit 14 carries out processing to actuate the notification unit 15 (step S103).

### <<Battery System>>

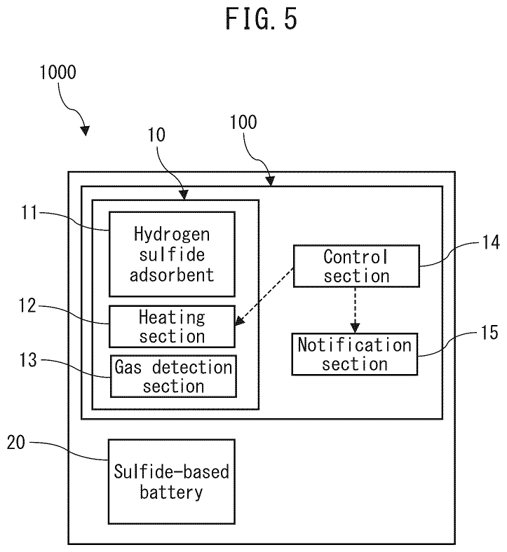

As shown in FIG. 5, the battery system 1000 of the disclosure comprises a sulfide-based battery 20 and a gas detection system 100 of the disclosure. In the battery system 1000 of the disclosure, hydrogen sulfide adsorbed by the hydrogen sulfide adsorbent 11 is generated from the sulfide-based battery. Specifically, the battery system 1000 of the disclosure comprises a sulfide-based battery 20 and a gas detection system 100, wherein the gas detection system comprises a hydrogen sulfide adsorbent 11 that adsorbs hydrogen sulfide generated from the sulfide-based battery and releases hydrogen sulfide and/or sulfur-containing gas derived from hydrogen sulfide at or above a predetermined temperature, a heating section 12 that heats the hydrogen sulfide adsorbent, a gas detection section 13 that detects hydrogen sulfide and/or sulfur-containing gas released from the hydrogen sulfide adsorbent, and a control unit 14 that actuates the heating section at the predetermined interval. With this construction, it is possible to detect with high precision hydrogen sulfide-containing gas that is sometimes generated from the sulfide-based battery 20, without depending on manual operation. The sulfide-based battery 20 will be understood by referring to the description herein regarding the battery pack of the disclosure. The gas detection system 100 will be understood by referring to the description herein regarding the gas detection system of the disclosure.

In the battery system 1000 of the disclosure, the sulfide-based battery 20 may be housed inside the outer container 30. The other components of the gas detection system 100, including the hydrogen sulfide adsorbent 11, the heating section 12 and the gas detection unit 10 comprising the gas detection section 13, may also be housed in the outer container 30. In other words, the construction of the battery system 1000 of the disclosure other than the control unit 14 and notification unit 15 may be the same construction as the battery pack 1 of the disclosure.

Processing carried out by the battery system of the disclosure may be the same as processing carried out by the gas detection system 100. Processing carried out by the battery system of the disclosure will therefore be understood by referring to FIG. 4 and the description herein regarding the gas detection system of the disclosure.

### First Application Example

A first application example of the battery system 1000 of the disclosure will now be described.

For the first application example, the battery system 1000 of the disclosure may comprise a battery pack 1 having a sulfide-based battery 20, a gas detection unit 10 as part of the gas detection system 100, and an outer container 30 housing them, as well as a control unit 14 and notification unit 15 forming the gas detection system 100. The battery pack may have an opening or discharge pathway allowing communication between the inside of the outer container and the outside of the battery pack.

According to the first application example, a hydrogen sulfide removing agent may be disposed inside the outer container or within the discharge pathway. When the hydrogen sulfide removing agent is disposed inside the outer container, the hydrogen sulfide removing agent may be housed in a hydrogen sulfide removing agent container connected with the discharge pathway. The hydrogen sulfide removing agent may also be disposed in a meandering flow passage inside the hydrogen sulfide removing agent container. The meandering flow passage can be formed by appropriate placement of a plurality of baffle plates having short lengths in the left-right direction with respect to the vertical cross-section in the direction of flow of hydrogen sulfide within the hydrogen sulfide removing agent container. The hydrogen sulfide removing agent is not particularly restricted so long as it is able to remove hydrogen sulfide. The material of the hydrogen sulfide removing agent may be the same as or different from the material of the hydrogen sulfide adsorbent.

When hydrogen sulfide has been generated from the sulfide-based battery in a battery system having such a construction, it is possible not only to detect hydrogen sulfide-containing gas at high precision with the gas detection section, but also to provide a notification with the notification unit, that hydrogen sulfide has been generated. The generated hydrogen sulfide can also be removed by the hydrogen sulfide removing agent. It is therefore possible for a user to recognize that hydrogen sulfide has been generated, and to effectively prevent leakage of generated hydrogen sulfide outside of the battery pack.

### Second Application Example

A second application example of the battery system 1000 of the disclosure will now be described.

For the second application example, similar to the first application example, the battery system 1000 of the disclosure may comprise a battery pack 1 having a sulfide-based battery 20, a gas detection unit 10 as part of the gas detection system 100, and an outer container 30 housing them, as well as a control unit 14 and notification unit 15 forming the gas detection system 100. The battery pack may have a circulation pathway for circulation of gas inside the outer container. In this case the battery pack may have a sealed construction.

When hydrogen sulfide has been generated from the sulfide-based battery in a battery system having such a construction, similar to the first application example, it is possible not only to detect hydrogen sulfide-containing gas at high precision with the gas detection section, but also to provide a notification with the notification unit 15, that hydrogen sulfide has been generated.

A program, prestored in the memory, may halt control by the control unit for actuation of the heating section, when the gas detection section has generated a signal for a detected concentration of hydrogen sulfide that is higher than the threshold and the signal has been inputted into the control unit, and the control unit may then halt control of actuation of the heating section based on the program. Such a construction will result in a final state with hydrogen sulfide adsorbed onto the hydrogen sulfide adsorbent. It is therefore possible to effectively prevent leakage of generated hydrogen sulfide outside of the battery pack.

For the first and second application examples, the battery pack may further comprise a ventilation section. The ventilation section is not particularly restricted so long as it generates a gas flow, and it may be a fan, for example. When the battery pack comprises a ventilation section, the ventilation section may be controlled by the control unit. Specifically, the ventilation section may be actuated when the concentration of the hydrogen sulfide and/or sulfur-containing gas as detected by the gas detection section is higher than the threshold, for example. With such a construction it is possible to increase efficiency for exhaust of gas in the battery pack for the first application example, and to increase efficiency for circulation of gas in the battery pack for the second application example.

### Third Application Example

A third application example of the battery system 1000 of the disclosure will now be described.

For the third application example, the battery system 1000 of the disclosure may be applied in a vehicle as a vehicle system. For the third application example, similar to the first and second application examples, the battery system 1000 of the disclosure may comprise a battery pack 1 having a sulfide-based battery 20, a gas detection unit 10 as part of the gas detection system 100, and an outer container 30 housing them, as well as a control unit 14 and notification unit 15 forming the gas detection system 100.

The battery pack may have an opening or discharge pathway allowing communication between the inside of the outer container and the outside of the vehicle, with the opening or discharge pathway being provided at a location where negative pressure is generated while the vehicle is traveling.

According to the third application example, a hydrogen sulfide removing agent may be disposed inside the outer container or within the discharge pathway. The case where the hydrogen sulfide removing agent is disposed inside the outer container may be understood by referring to the description of the first application example.

When hydrogen sulfide has been generated from the sulfide-based battery having such a construction, similar to the first and second application examples, it is possible not only to detect hydrogen sulfide-containing gas at high precision with the gas detection section, but also to provide a notification with the notification unit, that hydrogen sulfide has been generated. Since the opening or discharge pathway is provided at a location where negative pressure is generated while the vehicle is traveling, hydrogen sulfide-containing gas flows from the inside of the outer container where the pressure is relatively high, to the location outside of the vehicle where negative pressure is generated and the pressure is relatively low, thus allowing hydrogen sulfide-containing gas in the outer container to be discharged out of the vehicle via the hydrogen sulfide removing agent without the need for a ventilation section as in the first application example.

### <<Gas Detection Method>>

The method of the disclosure for detecting gas comprises the following steps: adsorbing hydrogen sulfide with a hydrogen sulfide adsorbent 11, heating the hydrogen sulfide adsorbent with a heating section 12 to cause release of the hydrogen sulfide and/or sulfur-containing gas derived from the hydrogen sulfide from the hydrogen sulfide adsorbent, and detecting the hydrogen sulfide and/or sulfur-containing gas released from the hydrogen sulfide adsorbent, with a gas detection section 13. This method allows detection of hydrogen sulfide-containing gas with high precision.

The method of the disclosure for detecting gas includes adsorbing hydrogen sulfide by the hydrogen sulfide adsorbent 11. The hydrogen sulfide adsorbent 11 will be understood by referring to the description herein regarding the gas detection unit of the disclosure.

The method of absorbing hydrogen sulfide with the hydrogen sulfide adsorbent is not particularly restricted, and an example is a method of contacting the hydrogen sulfide with the hydrogen sulfide adsorbent.

The method of the disclosure for detecting gas includes heating the hydrogen sulfide adsorbent with the heating section 12 to cause release of hydrogen sulfide and/or sulfur-containing gas derived from hydrogen sulfide, from the hydrogen sulfide adsorbent. The heating section 12 will be understood by referring to the description herein regarding the gas detection unit of the disclosure.

The method of heating the hydrogen sulfide adsorbent is not particularly restricted, and may be a conductive heating, convection heating or radiant heating method, for example. Heating of the hydrogen sulfide adsorbent causes release of hydrogen sulfide and/or sulfur-containing gas from the hydrogen sulfide adsorbent.

The method of the disclosure for detecting gas includes detecting hydrogen sulfide and/or sulfur-containing gas released from the hydrogen sulfide adsorbent, by the gas detection section 13. The gas detection section 13 will be understood by referring to the description herein regarding the gas detection unit of the disclosure.

The method for detecting hydrogen sulfide and/or sulfur-containing gas released from the hydrogen sulfide adsorbent by the gas detection section is not particularly restricted, and an example is a method of causing contacting of the gas detection section with the hydrogen sulfide and/or sulfur-containing gas released from the hydrogen sulfide adsorbent.

For the method of the disclosure, the hydrogen sulfide adsorption device may be disposed inside the outer container of the battery pack together with the sulfide-based battery, causing hydrogen sulfide generated from the sulfide-based battery to be adsorbed by the hydrogen sulfide adsorption device. The method allows high-precision detection of hydrogen sulfide that is often generated from a sulfide-based battery. The hydrogen sulfide adsorption device may comprise a gas detection unit of the disclosure.

### REFERENCE SIGNS LIST

1 Battery pack
10 Gas detection unit
11 Hydrogen sulfide adsorbent
12 Heating section
13 Gas detection section
14 Control unit
15 Notification unit
20 Sulfide-based battery
30 Outer container
100 Gas detection system
1000 Battery system

1. A gas detection unit comprising:
a hydrogen sulfide adsorbent that adsorbs hydrogen sulfide and releases the hydrogen sulfide and/or sulfur-containing gas derived from the hydrogen sulfide at or above a predetermined temperature,
a heating section that heats the hydrogen sulfide adsorbent, and
a gas detection section that detects the hydrogen sulfide and/or sulfur-containing gas released from the hydrogen sulfide adsorbent.

2. The gas detection unit according to claim 1, wherein the gas detection section is disposed above the hydrogen sulfide adsorbent.

3. The gas detection unit according to claim 1, wherein the heating section heats the hydrogen sulfide adsorbent at a temperature of 100° C. or higher and 400° C. or lower.

4. A battery pack comprising a sulfide-based battery, a gas detection unit according to claim 1, and an outer container that houses the sulfide-based battery and gas detection unit, wherein the hydrogen sulfide is generated from the sulfide-based battery.

5. The battery pack according to claim 4, wherein the hydrogen sulfide adsorbent is disposed below the sulfide-based battery.

6. A gas detection system comprising:
a gas detection unit according to claim 1, and
a control unit that actuates the heating section at a predetermined interval.

7. The gas detection system according to claim 6, wherein:
the gas detection system further comprises a notification unit,
the control unit actuates the notification unit when the concentration of the hydrogen sulfide and/or sulfur-containing gas detected by the gas detection section is higher than a threshold.

8. A battery system comprising a sulfide-based battery and a gas detection system according to claim 6,
wherein the hydrogen sulfide is generated from the sulfide-based battery.

9. A gas detection method comprising the following steps:
adsorbing hydrogen sulfide with a hydrogen sulfide adsorbent,
heating the hydrogen sulfide adsorbent with a heating section to cause release of the hydrogen sulfide and/or sulfur-containing gas derived from the hydrogen sulfide, from the hydrogen sulfide adsorbent, and
detecting the hydrogen sulfide and/or sulfur-containing gas released from the hydrogen sulfide adsorbent, with a gas detection section.

10. The gas detection method according to claim 9, wherein a hydrogen sulfide adsorption device is disposed in an outer container of a battery pack together with a sulfide-based battery, causing hydrogen sulfide generated from the sulfide-based battery to be adsorbed by the hydrogen sulfide adsorption device.
